(19) United States
(12) Patent Application Publication
(10) Pub. No.: US 2009/0105791 A1
(43) Pub. Date: Apr. 23, 2009

(54) LIGHT EMITTING MEDICAL GARMENT DEVICE, KIT AND METHOD OF USING

(76) Inventors: William J. McGINNIS, Cincinnati, OH (US); Scott A. METRICK, Glenview, IL (US)

Correspondence Address: LADAS & PARRY LLP, 224 SOUTH MICHIGAN AVENUE, SUITE 1600, CHICAGO, IL, 60604, US

(21) Appl. No.: 11/876,921

(22) Filed: Oct. 23, 2007

(51) Int. Cl. A61B 18/18

(52) U.S. Cl. 607/88

(57) ABSTRACT

A medical garment device is presented which is capable of providing at least two different energy light beams onto a therapeutic unhealthy zone of a patient. The device includes a first light source that can deliver a relatively low energy light beam, such as infrared radiation, for use in soothing and promoting healing of the therapeutic unhealthy zone. The device also includes a second light source that can deliver a relatively higher energy light beam, such as ultraviolet radiation, for use in discouraging growth of microorganisms. The device includes a base; a system on a chip (SOC) controlling a first and second bank of Light Emitting Diodes (LEDs), and a power unit powering the SOC and the first and second banks of LEDs. The kit includes the unassembled components of the device. The method includes the steps of affixing, assembling, attaching, obtaining, opening, and removing.

### FIELD OF THE INVENTION

The present invention relates to medical garments and accessories, more particularly to a light emitting medical garment device for use in providing at least two different energy light beams onto a therapeutic diseased zone of a patient.

### BACKGROUND

It has been known that the treatment of wounds, sores, and alike with the aid of light has a favorable effect on the healing processes, such as in accelerating healing. Infrared radiation is known to yield favorable effects on the healing processes of wounds, sores and alike. It is thought that infrared radiation not only provides soothing heat but it may also stimulate various sub cellular organelles, such as mitochondria, to promote healing bio-activities. Ultraviolet (UV) radiation is also known to yield favorable effects on the healing of wounds, sores and alike. One well known example is that UV radiation is known to promote the healing of hyperbilirubinemia (jaundice) which is common in infants, and affect, in some degree, up to half of full-term infants and most of preterm infants.

Therefore, a need exists for a new and improved light emitting medical garment device capable of providing at least two different energy light beams onto a therapeutic diseased zone. In this respect, the light emitting medical garment device according to the present invention substantially departs from the conventional concepts and designs of the prior art, and in doing so provides an apparatus primarily developed for the purpose of providing a convenient means for making it possible to provide at least two different energy light beams onto a therapeutic diseased zone.

### SUMMARY OF THE INVENTION

The present device, kit and method of using same, according to the principles of the present invention provides a novel an non-obvious medical garment device that is capable of providing at least two different energy light beams onto a therapeutic unhealthy zone of a patient. The device includes a first light source that can deliver a relatively low energy light beam, such as infrared radiation, for use in soothing and promoting healing of the therapeutic unhealthy zone. The device also includes a second light source that can deliver a relatively higher energy light beam, such as ultraviolet radiation, for use in discouraging growth of microorganisms. The device includes a base; a system on a chip (SOC) controlling a first and second bank of Light Emitting Diodes (LEDs), and a power unit powering the SOC and the first and second banks of LEDs. The kit includes the unassembled components of the device. The method includes the steps of affixing, assembling, attaching, obtaining, opening, and removing.

The present invention provides an improved light emitting medical garment device, which will be described subsequently in great detail. The new and improved light emitting medical garment device is not anticipated, rendered obvious, suggested, or even implied by the prior art, either alone or in any combination thereof.

To attain this, the present invention essentially comprises light emitting medical garment device comprises a base; a system on a chip (SOC) controlling a first and second bank of Light Emitting Diodes (LEDs), and a power unit powering the SOC and the first and second banks of LEDs. The light emitting medical garment device is capable of providing at least two different energy light beams onto a therapeutic unhealthy zone of a patient. The device includes a first light source that can deliver a relatively low energy light beam, such as infrared radiation, for use in soothing and promoting healing of the therapeutic unhealthy zone. The device also includes a second light source that can deliver a relatively higher energy light beam, such as ultraviolet radiation, for use in discouraging growth of microorganisms.

The kit includes the unassembled components of the device. The method includes the steps of affixing, assembling, attaching, obtaining, opening, and removing.

There has thus been outlined, rather broadly, the more important features of the invention in order that the detailed description thereof that follows may be better understood, and in order that the present contribution of the art may be better appreciated.

The invention may also include an optional container for enshrouding the device to maintain the sterility of the device.

Numerous features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Even still another aspect of the present invention is to provide a light emitting medical garment device having base; a first and second bank of LEDs; a SOC, and a power unit in which the second spectrum of light emanating from the second LED has a energy greater than the first sepectrum of light emanating from the first LED

Still another aspect of the present invention is to provide a kit comprising the unassembled components of the device.

Lastly, it is an object of the present invention to provide a new and improved method of using comprising the steps of affixing, assembling, attaching, obtaining, opening, and removing.

Unless otherwise defined, all scientific and technical terms used herein are to be construed as having the same meaning as commonly understood by one of ordinary skill in the art to which this invention pertains. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present document, including definitions, will control. Unless otherwise indicated, materials, methods, and examples described herein are illustrative only and not intended to be limiting.

There has thus been outlined, rather broadly, the more important features of the invention in order that the detailed description thereof that follows may be better understood, and in order that the present contribution of the art may be better appreciated.

Numerous other features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIGS. 1A and 1B depict a respective bottom and top plan view of an embodiment of the light emitting medical garment device constructed in accordance with the principles of the present invention;

FIG. 2 depicts a cross sectional view along of one embodiment of the light emitting medical garment device of the present invention;

FIGS. 3A, 3B, 3C, and 3D depict cross sectional view of various embodiments of the light emitting medical garment device of the present invention;

FIGS. 4A, 4B, and 4C depict perspective views of various embodiments of the light emitting medical garment device of the present invention;

FIG. 5 depicts a block diagram of one embodiment of the light emitting medical garment device of the present invention;

FIGS. 6A, 6B, 6C, 6D, 6E, and 6F depict various embodiments of the first and second timed on/off sequences imposed on the respective first and second banks of LEDs of the light emitting medical garment device of the present invention;

FIGS. 7A, 7B, 7C, 7D, 7E, and 7F depict various embodiments of the how the first and second banks of LEDs are electronically connected together; and

FIGS. 8A, 8B, 8C, 8D, 8E, 8F, 8G, 8H, 8I, 8J, 8K, 8L, 8M, 8N, and 8O depict various configurations of the light emitting medical garment device of the present invention.

The same reference numerals refer to the same parts throughout the various figures.

### DETAILED DESCRIPTION OF THE PREFERRED EMBODIMENT

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

It will be understood that when a layer is referred to as being “on” another layer or substrate, it can be directly on the other layer or substrate, or intervening layers may also be present. In the drawings, the thickness of layers and regions are exaggerated for clarity and like reference numerals denote like elements.

Accordingly, the detailed discussion herein of one or more embodiments is not intended, nor is to be construed, to limit the metes and bounds of the patent protection afforded the present invention, in which the scope of patent protection is intended to be defined by the claims and their equivalents thereof. Therefore, embodiments not specifically addressed herein, such as adaptations, variations, modifications, and equivalent arrangements, should be and are considered to be implicitly disclosed by the illustrative embodiments and claims described herein and therefore fall within the scope of the present invention.

Further, it should be understood that, although steps of various the claimed method may be shown and described as being in a sequence or temporal order, the steps of any such method are not limited to being carried out in any particular sequence or order, absent an indication otherwise. That is, the claimed method steps are to be considered to be capable of being carried out in any sequential combination or permutation order while still falling within the scope of the present invention.

Additionally, it is important to note that each term used herein refers to that which a person skilled in the relevant art would understand such term to mean based on the contextual use of such term herein. To the extent that the meaning of a term used herein, as understood by the person skilled in the relevant art based on the contextual use of such term, differs in any way from any particular dictionary definition of such term, it is intended that the meaning of the term as understood by the person skilled in the relevant art should prevail.

It is understood in the contexts of the subject of this invention that every light source can be functionally described by its spectral emission of light within a particular range of wavelengths. It is also understood, that monochromatic light is defined as special type of light having only a single specific wavelength. Light emitting diodes can be configured to emit relatively narrow to relatively broad spectral bandwidths in which the emitted spectra may have localized minima and maxima wavelengths.

The term “bandwidth” is understood to refer to a lower and a upper wavelength limit defining the outer bounds of an emission spectrum sets at some arbitrary limit, say a limit of about 5% power intensity relative to the power intensity of the predominant wavelength within that emission spectrum.

The term “photosensitizer active agent” in the context of the subject invention refers to any compound that is capable of absorbing electromagnetic (EMF) radiation (usually from the light spectral frequencies) and subsequently converting this absorbed EMF radiation into a chemical excited reactive state. This excited reactive states of the photosensitizer active agent may be any physical or physiochemical excited state such as promoting the photosensitizer active agent into a singlet excited state, a triplet excited state, a pi-pi* excited state, or a intermediate excited state transferring a charge to an another molecule. Photosensitizer active agents typically have chemical bond structures that include multiple conjugated rings that allow for the light absorption and photoactivation. The Photosensitizer active agents may be involved in any number of different chemical mechanisms including fluorescence mechanisms, chemical phosphorescence mechanisms, free radical generating mechanisms, and charge transfer mechanisms. Accordingly, these types of light activated excited states of photosensitizer active agents may result in generating nitrogen oxide, singlet oxygen, ozone, free radicals and charged chemical carriers within the tissue. Once excited, these photosensitizer active agents may contribute to lowering the population densities of prokayotic and eukaryotic cells such as bacteria, mycoplasmas and yeast. Preferably, the photosensitizer active agent is essentially nontoxic, except for the desired cytotoxic effect produced locally, upon photoactivation of the photosensitizer active agent.

The term “therapeutic site” is understood to refer to any site in need a therapeutic care from a physician, such as, a wound, an incision, a scare, a wart (recalcitrant, verruca vulgaris or verruca plantaris), a port-wine stain (naevus flammeus), a jaundice individual (such as an infant or an individual inflicted with hepatitis), or any zone on a body zone diseased with acne, seborrhoea, eczema, psoriasis, nevus sebacous, squamous cell carcinoma, intraepithelial carcinoma, mycosis gunoides, warts (recalcitrant, verruca vulgaris or verruca plantaris), toxoplasma, listeria, salmonella, or leishmania.

The term “pharmaceutical acceptable salts” is understood to include counter-anionic species, but are not limited to, halides (e.g., fluoride, chloride, bromide and iodide), sulfates (e.g., decylsulfate), nitrates, perchlorates, sulfonates (e.g., methan sulfonate) and trifluoroacetate. Also “pharmaceutical acceptable salts” may include acid addition salts, for example salts formed with inorganic acids, such a hydrochloric, hydrobromic, sulfuric, sulfonic, phosphoric, carboxylic, organo-sulfonic acids. Also “pharmaceutical acceptable salts” may include counter-cationic species, but are not limited to, lithium, sodium, potassium, calcium, magnesium, ammonium, hydroxyethyl ammonium, piperdine, and pyridinium salts.

Furthermore, a person skilled in the art of reading claimed inventions should understand that “a” and “an” each generally denotes “at least one,” but does not exclude a plurality unless the contextual use dictates otherwise. And that the term “or” denotes “at least one of the items,” but does not exclude a plurality of items of the list.

The following detailed embodiments presented herein are for illustrative purposes. That is, these detailed embodiments are intended to be exemplary of the present invention for the purposes of providing and aiding a person skilled in the pertinent art to readily understand how to make and use of the present invention.

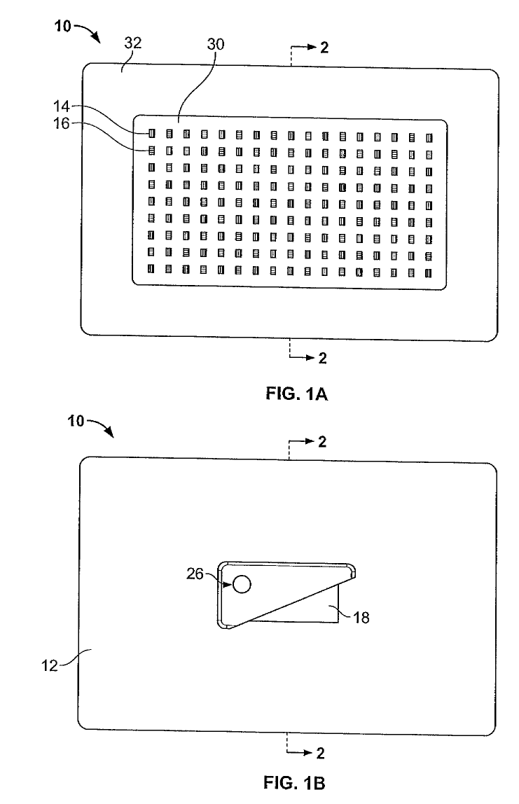

Referring now to the drawings, and in particular FIGS. 1 to 8 thereof, one preferred embodiment of the present invention is shown and generally designated by the reference numeral 10. The same reference numerals refer to the same parts throughout the various figures.

One preferred embodiment of a light emitting medical garment device 10 comprises: a base 12; a first bank Light Emitting Diodes (LEDs) 14, a second bank of LEDs 16 16; a System On a Chip (SOC) 18; and a power unit 20. The first bank of LEDs is attached to the base 12 wherein the first bank of LEDs being configured to emit a first spectrum of light, wherein the first spectrum of light being defined by a first spectral bandwidth of wavelengths and having a first predominant wavelength. The second bank of LEDs 16 is attached to the base 12 wherein the second bank of LEDs 16 is configured to emit a second spectrum of light. The second spectrum of light being defined by a second spectral bandwidth of wavelengths and having a second predominant wavelength wherein the second predominant wavelength having an energy greater than the first predominant wavelength. The SOC 18 is electrically coupled to the first and second banks of LEDs (14 and 16, respectively). The power unit 20 is operatively coupled to the SOC 18, and is operatively coupled to the first and second banks of LEDs (14 and 16, respectively).

The base 12 of the light emitting medical garment device 10 may be any known design and can include an inner pad 30, an outer coating 32, and an adhesive layer 34 connecting the inner pad 30 to the outer coat.

The power unit 20 of the light emitting medical garment device 10 may be any commercially available power unit 20 such as any of selected from the group, but not limited to, the group consisting of a battery power unit 20, a high capacity capacitor power unit 20, a transformer power unit 20, and an electrical outlet plug power unit 20.

The first bank of LEDs may be any commercially known LED configured to emit light between about 1000 nm to about 570 nm (infrared to orange). One suitable LED design making up the first bank of LEDs may be the LED base 12d on aluminum indium gallium phosphide/gallium phosphide (AlInGaP/GaP) diode design that can be configured to provide output wavelengths in a range between about 590 to about 640 nm with peak emission wavelengths of about 590 nm, 596 nm, 605 nm, 615 nm, 626 nm, 630 nm, and 640 nm. Another suitable LED design making up the first bank of LEDs may be the aluminium indium gallium phosphide/gallium arsenic (AlInGaP/GaAs) diode designs that can be configured to provide output wavelengths in a range between about 560 to about 644 nm with peak emission wavelengths of about 562 nm, 574 nm, 612 nm, 620 nm, 623 nm and 644 nm. Yet another suitable LED design making up the first bank of LEDs can be the aluminum gallium arsenic (AlGaAs) diode designs that can be configured to provide output wavelengths in a range between about 650 nm to about 660 nm. Still another suitable first LED base 12d on the gallium phosphide (GaP) diode that can be configured to provide output wavelengths in a range between about 550 to 570 nm. Still yet another suitable LED design making up the first bank of LEDs can be the n-type gallium arsenide/p-type gallium arsenide (n-GaAs/p-GaAs) diode designs that can be configured to provide wavelengths in a range between about 900 nm to about 980 nm. Even yet another suitable LED design making up the first bank of LEDs can be the gallium aluminum arsenide diode designs that can be configured to provide wavelengths in a range between about 940 nm to about 945 nm.

The first bank of LEDs is preferred to be configured to emit light having a first luminescent power density of between about 1 μW/cm² to about 1 W/cm². The first bank of LEDs is preferred to be configured to emit light at a first predominant wavelength having a first bandwidth between about 1 nm to about 200 nm. The first predominant wavelength is preferred to be between about 1000 nm to about 570 nm.

The second bank of LEDs 16 may be any commercially known LED configured to emit light between about 570 nm to about 350 nm (yellow to ultraviolet). One suitable LED design making up the second bank of LEDs 16 can be those of the indium gallium nitride (InGaN) diode designs that can be configured to provide output wavelengths in a range between about 445 to about 530 nm with peak emission wavelengths of about 470 nm, about 505 nm and about 525 nm. Another suitable LED design making up the second bank of LEDs 16 can be those of the gallium nitride/silicon (GaN/Si) diode designs that can be configured to provide output wavelength of about 430 nm. Yet another suitable LED design making up the second bank of LEDs 16 can be those of zinc oxide (ZnO, ZnS, ZnSe, TiO₂) diode designs that can be configured to provide output wavelengths between about 380 nm to about 500 nm. Yet another suitable LED design making up the second bank of LEDs 16 can be those of gallium nitride (GaN) diode designs encompassing the InGaN, AlGaN and AlInGaN diode designs that can be configured to provide output wavelengths between about 420 to about 470 nm.

The second bank of LEDs 16 is preferred to be configured to emit light having a second luminescent power density between about 1 μW/cm² to about 1 W/cm². The second bank of LEDs 16 is preferred to be configured to emit light having a second bandwidth between about 1 nm to about 200 nm. The second predominant wavelength is preferred to be between about 570 nm to about 350 nm.

The SOC 18 of the of the light emitting medical garment device 10 may be is attached to the base 12 or it may be attached to the external housing 24. The SOC 18 may optionally be electronically configured to direct the first and second banks of LEDs (14 and 16, respectively) in any sequence, such as, being constantly on as long as the power unit 20 supplies power. Another embodiment of the SOC 18 is that the SOC 18 is configured to direct the first bank of LEDs in accordance to a first timed on/off sequence 36, and the SOC 18 is configured to direct the second bank of LEDs 16 in accordance to a second timed on/off sequence 38.

The power unit 20 of the light emitting medical garment device 10 may be is attached to the base 12 or may be attached to the external housing 24.

An optional container 22 may be added to the light emitting medical garment device 10 in which the container 22 enshrouds or envelopes the device 10.

An optional external housing 24 may be added to the light emitting medical garment device 10 in which the external housing 24 is attached to the power unit 20.

An optional ON/OFF switch 26 may be added to the light emitting medical garment device 10 in which the ON/OFF switch 26 is electrically coupled to the SOC 18.

An optional an active agent 28 may be added to the light emitting medical garment device 10 in which the active agent 28 is attached to the base 12. The active agent 28 may be selected from the group consisting of an anti-allergy active agent 28, an analgesic active agent 28, an anesthetic active agent 28, an antibiotic active agent 28, an anti-eczema active agent 28, an antifungal active agent 28, an anti-inflammatory active agent 28, an antiseptic active agent 28, an antiviral active agent 28, a disinfectant active agent 28, an immunosuppressant active agent 28, an steroidal active agent 28, a photosensitizer active agent 28, and admixtures thereof.

The antibiotic embodiment of the active agent 28 may be selected from the group, but not limited to, consisting of the antibiotic is selected from the group consisting of alborixin, amikacin, amphotericin B, ampicillin, aureofungin, bacitracin, bekanamycin, calcimycin, carbenicillin, cephalothin, chloramphenicol. chlortetracycline, chloromycetin. clindamycin, colistimethate, colistin, demeclocycline, dianemycin, dibekacin, doxycycline, econazole, etheromycin, erythromycin, gentamicin, grisorixin, ionomycin, kanamycin, laidlomycin, lasalocid, lenoremycin, lincomycin, lonomycin, lucensomycin, lymecycline, meclocycline, methyacycline, minocycline, monensin, monensin phenylurethane derivatives, mutalamycin, nalidixic Acid, narasin, natamycin, N-demethylrifampicin, neomycin, nigericin, nitrofurantoin, novobiocin, nystatin, oxytetracycline, penicillin, polyamido streptomycin, polymyxin, rifampicin, rolitetracycline, salinomycin, septamycin, streptomycin, streptolydigin, tetracycline, tobramycin, trimethoprim-sulfamethoxazole, and their pharmaceutically acceptable salts.

The antifungal embodiment of the active agent 28 may be selected from the group, but not limited to, consisting of 1-(4chlorophenoxy)-1-(1H-imidazolyl)-3,3-dimethyl-2-butanone, 6-cyclohexyl-1-hydroxy-4methyl-2(1H)-pyridone, 1-hydroxy-4-methyl-6-(2,4,4trimethylpentyl)-2-(1H)-pyridone, acyclovir, amorolfine, amphotericin B, azoles, benzyl peroxide, bifonazole, butenafine, butoconazole, chlorbutanol, chloroxylenol, ciclopirox, ciclopirox olaimine, cis-1-Acetyl-4[4-[[2-(2,4-dichlorophenyl)-2-(1H-imidazol-1-ylrnethyl)-1,3-dioxolan-4yl]methoxy]phenyl]piperazine, clotrimazole, clotrizole, diazoles, econazole, fluconazole, griseofulvin, iSOC 18onazole, itraconazole, itraconazole griseofulvin, ketoconazole, miconazole, nystatin, oxiconazole, povidone, saclicylic acid, saperconazole, sperconazole, sulconazole, 1H-1,2,4-triazole-1-ethanol, terbinafine, terconazole, tioconazole, tolnaftate, triazoles, undecylenic acid, voriconazole and their pharmaceutically acceptable salts.

The scabicide embodiment of the active agent 28 may be selected from the group, but not limited to, consisting of benzyl benzoate, crotamiton and malathion and their pharmaceutically acceptable salts.

The disinfectant embodiment of the active agent 28 may be selected from the group, but not limited to, consisting of 5-Chloro-2-(2,4-dichlorophenoxy)phenol, 1-hexadecylpyridinium chloride, N N″-bis(4-chlorophenyl)-3,12-diimino-2,4,11,13-tetraaza tetradecane diimidamide, 1, I′-hexamethylenebis[5-(p-chlorophenyl)biguanide], camphor, centrimide, chloramine, chlorhexidine, citric acid, electron deficient quinones, ethyl alcohol, ethylenediamine tetraacetate, hydrogen peroxide, hypochlorite, isopropyl alcohol, lactic acid, menthol, o-phenylphenol, o-benzyl-o-cholorophenol, percarbonate, permanganate, persulfate, povidone-iodine, urea peroxides, xylenol, and their pharmaceutically acceptable salts.

The antiviral embodiment of the active agent 28 may be selected from the group, but not limited to, consisting of acyclovir, amantadine, 2-amino-1,9-dihydro-9-[(2-hydroxyethoxy)methyl1]6H-purin-6-one, dideoxyuridine, azothymidine, cis-1-acetyl-4-[4-[[2-(2,4-dichlorophenyl)-2-(1H-imidazol-1-ylmethyl)-1,3-dioxo-1 an-4-yl]methoxy]-phenyl]piperazine, didanosine, flucytosine (5FC), foscarnet, ganeiclovir, interferon, larniyudine, phenol, ribavirin, ritonavir, stayudine, vidarabine, zalcitabine, zidovudine, and their pharmaceutically acceptable salts.

The anti-inflammatory embodiment of the active agent 28 may be selected from the group, but not limited to, consisting of cortisone, dexamethasone, diclofenac, diflunisal, etodolac, fenoproben, flunisolide, hydrocortisone, ibuprofen, indomethacin, ketoprofen, ketorolac tromethamine, meclofenamate, mefenamic acid, mesalamine, methyl prednisolone, nabumetone, naproxen, piroxicam, rednisolone, prednisone, salsalate, salicylsalicylic acid, sulindac, triamcinoline, and their pharmaceutically acceptable salts.

The anesthetic embodiment of the embodiment of the active agent 28 may be selected from the group, but not limited to, consisting of articaine, benzocaine, bupivacaine, bupivicaine, carbocaine, chloroprocaine, dibucaine, dimethylacetanilide, diphenhydramine, dyclonine, etidocaine, ethyl aminobenzoate, isobucaine, ketocaine, kinizocaine, lidocaine, lignocaine, marcaine, mepivacaine, meprylcaine, omega-diethylamino-2,6- and 4-aminobenzoic acid ethyl ester, parethoxycaine, piperocaine, prilocaine, primacaine, procaine, proparacaine, propoxycaine, pyrrocaine, rodocaine, ropivacaine, ropivacaine, tetracaine, trimecaine, xylocaine, and their pharmaceutically acceptable salts.

The steroidal embodiment of the active agent 28 may be selected from the group, but not limited to, consisting of acetonide, aclometasone, amcinonide, beclometasone, betamethasone, clobetasol, clobetasone, clocortolone, cortisone, desonide, desoxitnethasone, dexamethasone, diflorasone, diflucortolone, fluocinolone, fluocinonide, flumethasone, flurandrenolide, fluticasone, halcinonide, halobetasol, hydrocortisone, methylprednisolone, mometasone furoate, pivolate, prednicarbate, prednisolone, prednisone, triamcinolone and their pharmaceutically acceptable salts.

The anti-eczema embodiment of the active agent 28 may be selected from the group, but not limited to, consisting of calcipotriol, dithranolsalicylic acid, gamolenic acid, lithium succinate, tacalciol, tazarotene, and their pharmaceutically acceptable salts.

The antiseptic embodiment of the active agent 28 may be selected from the group, but not limited to, consisting of benzalkonium chloride, benzocaine, benzoic acid, benzoyl peroxide, cetrimide, cetylpyridinium, chlorbutanol, chlorhexidine, chlorhexideine gluconate, chlorocresol, chlorotetracycline, chloroxylenol, colistin, dibromopropamidine isothionate, framycetin, fusidic acid, hexachloropentane, hexachlorophene, hexetidine, hydroxyquinalone, iodine, lidocaine, methyl salicylate, metronidazole, muprocin, neomycin, nitrofurazone, phenol, polymoxin, povidone, resorcinol, silver sulfadiazine, tetracycline, triclosan, and their pharmaceutically acceptable salts.

The anti-allergy embodiment of the active agent 28 may be selected from the group, but not limited to, consisting of aluminum sulphate, antazoline, benzocaine, chlorbutanol, crotamiton, diphenhydramine, hydrocortisone, lignocaine, mepyramine, triclosan, and their pharmaceutically acceptable salts; the immunosuppressant active agent 28 is selected from the group consisting of ascomycin, azathioprine, etamethasone diproprionate, betamethasone valerate, clobetasol proprionate, cyclosporin A, cyclosporin B, cyclosporin G, fluocinolone acetonide, halcinonide, halobetasol proprionate, hexachlorobenzene, hydrocortisone, hydrocortisone valerate, rapamycin, tacrolimus, triamcinolone acetonide, and their pharmaceutically acceptable salts.

The photosensitizer embodiment of the active agent 28 may be selected from the group, but not limited to, consisting of acridine dyes, Azure A, Azure B, Azure C, antroquinones, bacteriochlorins, basic fuschin, benzophorphyrins, Brilliant Green, carbon black, chlorins, courmarins, Crystal Violet, flavin dyes, indocyanine green, Janus Green, Malachite Green, methylene blue, methyl green, m-tetrahydroxyphyenyl chlorin, naphthalocyanines, neural red dye, new fuschin, N-hydroxypyridine-2-(1H)-thione, pararosaniline acetate, patent blue VF, phthalocyanines, pheophorbides, phenothiazines, porphyrins, psoralens, purpurins, quinolones, quinones, riboflavin-5-phosphate, Rose Bengal, tetrapyrroles, texaphyrins, tinetiopurpurins, toluidine dyes, tri-arylmethane dyes, verdins, and pharmaceutical acceptable salts.

The form that the light emitting medical garment device 10 may be formed in any known medical garment device 10 design such as being formed in the group consisting of a bandage medical garment device 10, a blanket medical garment device 10, a scarf medical garment device 10, a neck brace medical garment device 10, a back brace medical garment device 10, a knee brace medical garment device 10, an ankle brace medical garment device 10, a SOC 18 k medical garment device 10, a glove medical garment device 10, a mitten medical garment device 10, a finger cot medical garment device 10, a hat medical garment device 10, a shirt medical garment device 10, a brassiere medical garment device 10 and an eye-patch sleeve medical garment device 10.

One preferred embodiment of the kit for the light emitting medical garment device 10 comprises: a base 12; a first bank of LEDs 14, a second bank of LEDs 16, a SOC 18, and a power unit 20. The first bank of LEDs 14 is attached to the base 12 in which the first bank of LEDs 14 is configured to emit a first spectrum of light, wherein the first spectrum of light is defined by a first spectral bandwidth of wavelengths and having a first predominant wavelength. The second bank of LEDs 16 is attached to the base 12 in which the second bank of LEDs 16 is configured to emit a first spectrum of light, wherein the second spectrum of light being defined by a second spectral bandwidth of wavelengths and having a second predominant wavelength, wherein the second predominant wavelength having an energy greater than the first predominant wavelength. The SOC 18 is electrically coupled to the first and second banks of LEDs (14 and 16, respectively). The power unit 20 is operatively attachable to the SOC 18, and operatively attachable to the first and second banks of LEDs (14 and 16, respectively).

An optional container 22 may be added to the kit in which the optional container 22 enshrouding the kit.

An optional external housing 24 may be added to the kit in which the optional external housing 24 is attachable to the power unit 20 and attachable to the SOC 18.

An optional active agent 28 may be added to the kit in which the optional active agent 28 is attachable to the base 12.

One embodiment of a method of using a kit for a light emitting medical garment device 10, the method comprising the steps of affixing, assembling, attaching, obtaining, opening, and removing.

The obtaining step comprises obtaining the kit comprising: a base 12; a first bank LEDs 14 is attached to the base 12 wherein the first bank of LEDs 14 is configured to emit a first spectrum of light, wherein the first spectrum of light being defined by a first spectral bandwidth of wavelengths and having a first predominant wavelength; a second bank of LEDs 16 is attached to the base 12 wherein the second bank of LEDs 16 is configured to emit a second spectrum of light, wherein the second spectrum of light being defined by a second spectral bandwidth of wavelengths and having a second predominant wavelength, wherein the second predominant wavelength having an energy greater than the first predominant wavelength; a SOC 18 is electrically coupled to the first and second banks of LEDs (14 and 16, respectively); a container 22 enshrouding the base 12, and the first and second banks of LED, and the SOC 18; a power unit 20 is operatively attachable to the SOC 18, and operatively attachable to the first and second banks of LEDs (14 and 16, respectively); and an active agent 28 is attachable to the base 12. The opening step comprises opening up the container 22 enshrouding the base 12, and the first and second banks of LEDs (14 and 16, respectively), and the SOC 18. The removing step comprises removing the base 12 and the first and second banks of LEDs (14 and 16, respectively) and the SOC 18 from the container 22. The assembling step comprises assembling the device 10 by coupling together the power unit 20 to the SOC 18 to enable the first and second banks of LEDs (14 and 16, respectively) to emit light. The affixing step comprises affixing a portion of the active agent 28 either onto the base 12 or onto a therapeutic site of a patient 40. The attaching step comprises attaching the device 10 onto the therapeutic site of the patient 40.

Referring now to FIG. 1A that depicts a bottom view of an embodiment of the light emitting medical garment device 10 showing the base 12 composed of an inner pad 30 and an outer coating 32; a first and second bank of LEDs (14 and 16, respectively).

Referring now to FIG. 1B that depicts a top view of an embodiment of the light emitting medical garment device 10 showing the base 12, the SOC 18 and the ON/OFF switch 26.

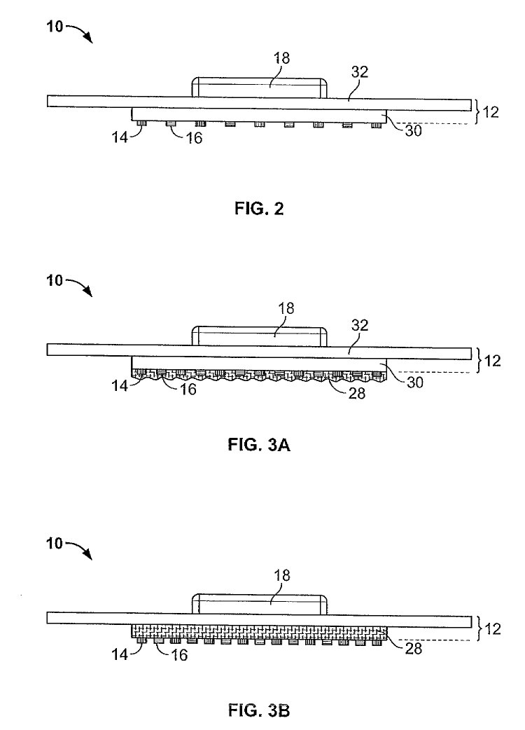

Referring now to FIG. 2 that depicts a cross sectional view along of one embodiment of the light emitting medical garment device 10 showing the base 12 composed of an inner pad 30 and an outer coating 32; a first and second bank of LEDs (14 and 16, respectively) mounted to the inner pad 30, and the SOC 18 mounted on the outer coating 32.

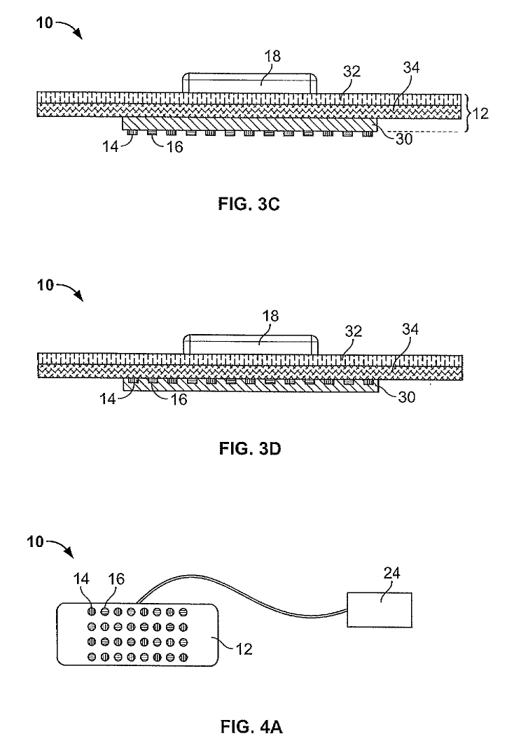

Referring now to FIGS. 3A, 3B, 3C, and 3D that depict cross sectional views of various embodiments of the light emitting medical garment device 10 showing the base 12 composed of an inner pad 30, an outer coating 32 and an adhesive layer 34; a first and second bank of LEDs (14 and 16, respectively) mounted to the inner pad 30 of the base 12, the SOC 18 mounted on the outer coating 32 and the active agent 28 attached to the inner pad 30 and to the first and second bank of LEDs (14 and 16, respectively).

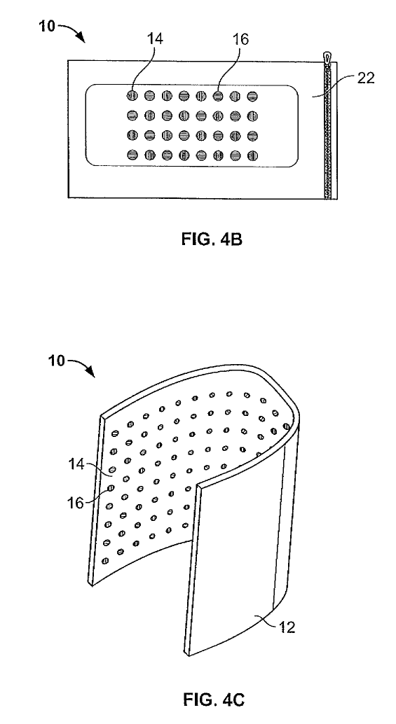

Referring now to FIGS. 4A, 4B, and 4C depict perspective views of various embodiments of the light emitting medical garment device 10 showing the a first and second bank of LEDs (14 and 16, respectively) attached to the base 12, the external housing 24 attached to the base 12, and the container 22 enveloping the device 10.

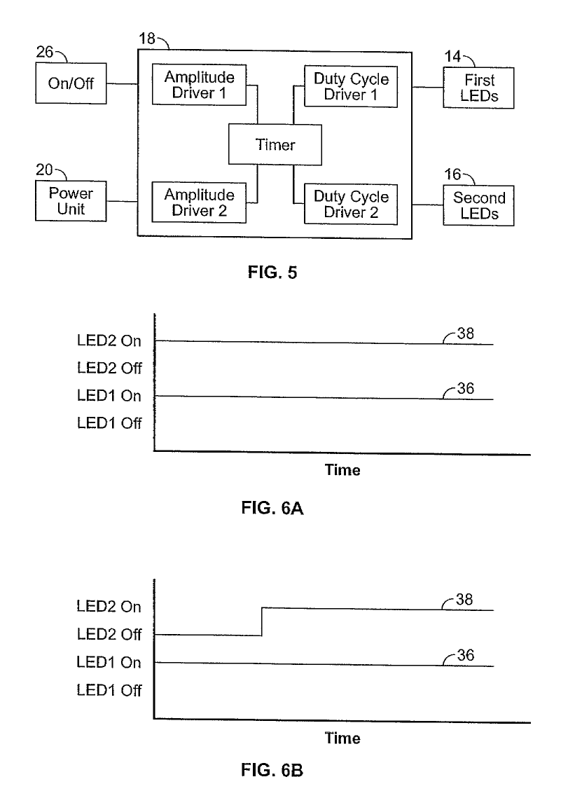

Referring now to FIG. 5 that depicts a block diagram of one embodiment of the light emitting medical garment device 10 showing the SOC 18 operatively coupled to the power unit 20, the ON/OFF switch 26, and the first and second banks of LEDs (14 and 16, respectively). Within the SOC 18 is shown a first amplifier driver and a first duty cycle driver for independently controlling the first bank of LEDs 14. Also within the SOC 18 is shown a second amplifier driver and a second duty cycle driver for independently controlling the second bank of LEDs 16.

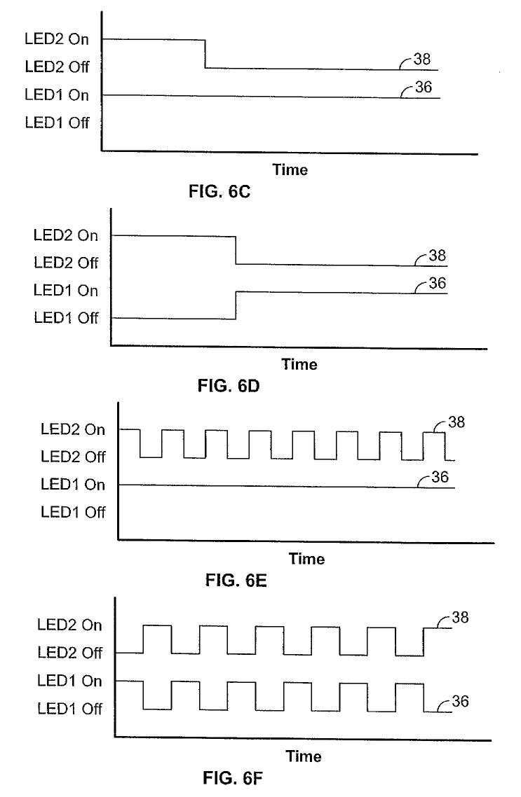

Referring now to FIGS. 6A, 6B, 6C, 6D, 6E, and 6F that depict various timing sequence embodiments of how the SOC 18 can control the timed on/off sequences imposed on the respective first and second banks of LEDs (14 and 16, respectively) of the light emitting medical garment device 10.

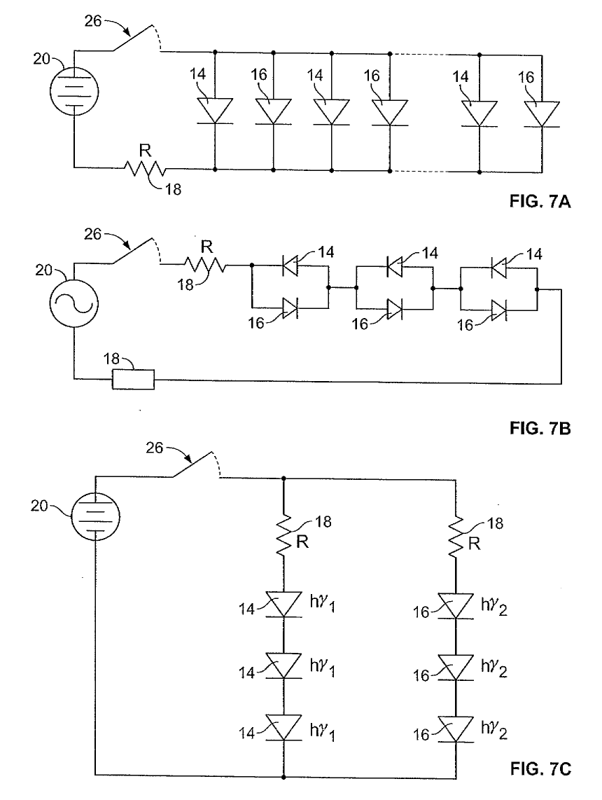

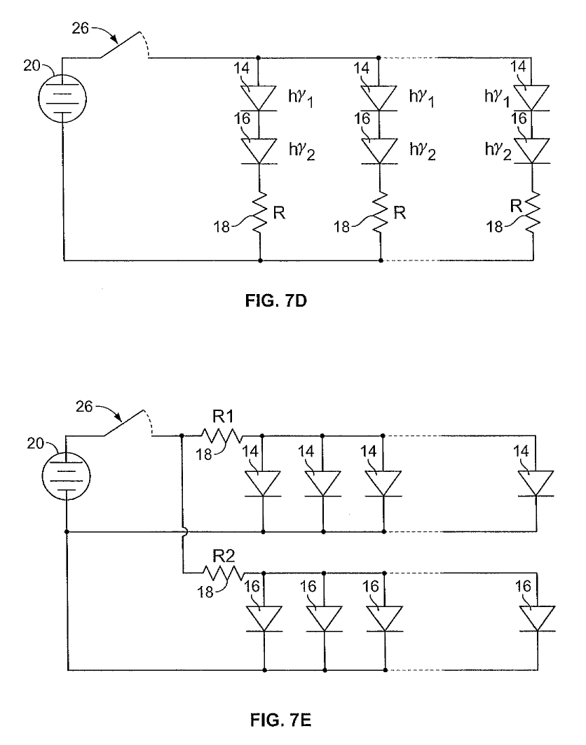

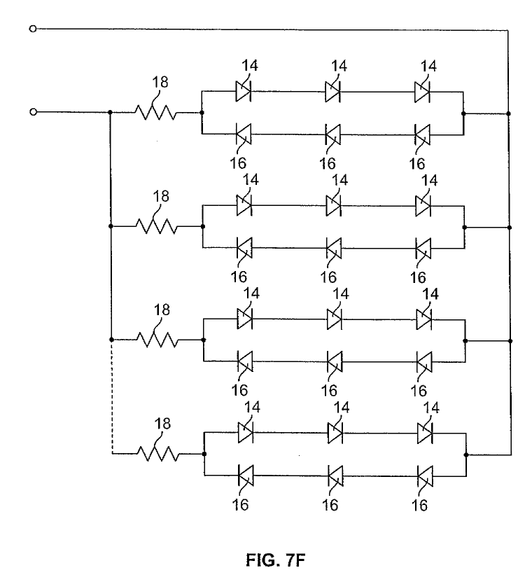

Referring now to FIGS. 7A, 7B, 7C, 7D, 7E, and 7F depict various embodiments, but not limited to, of the how the first and second banks of LEDs (14 and 16, respectively) can be electronically connected together.

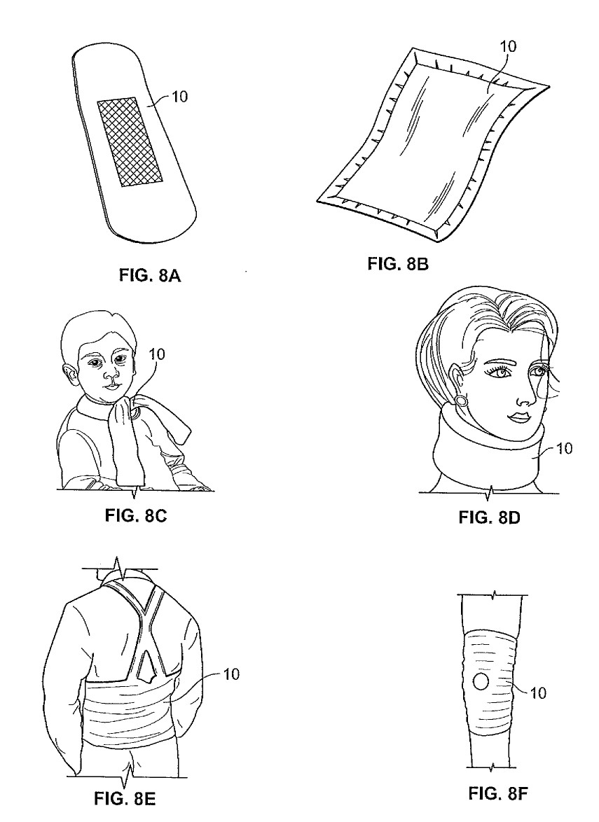

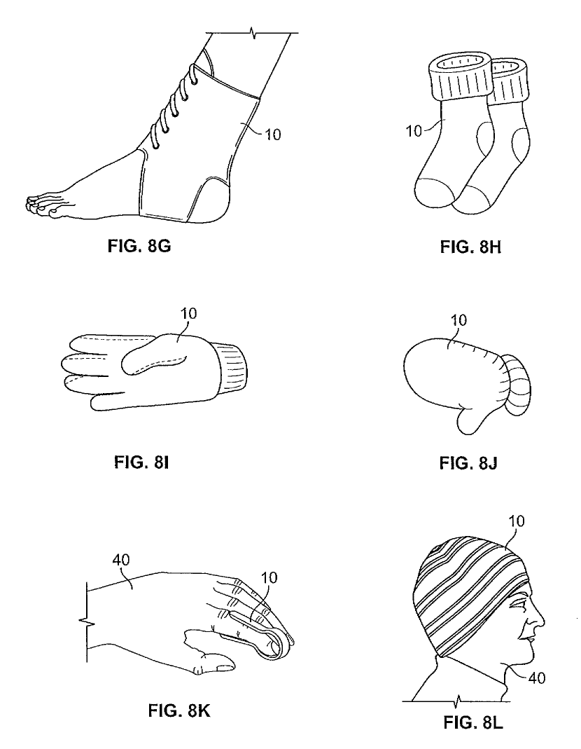

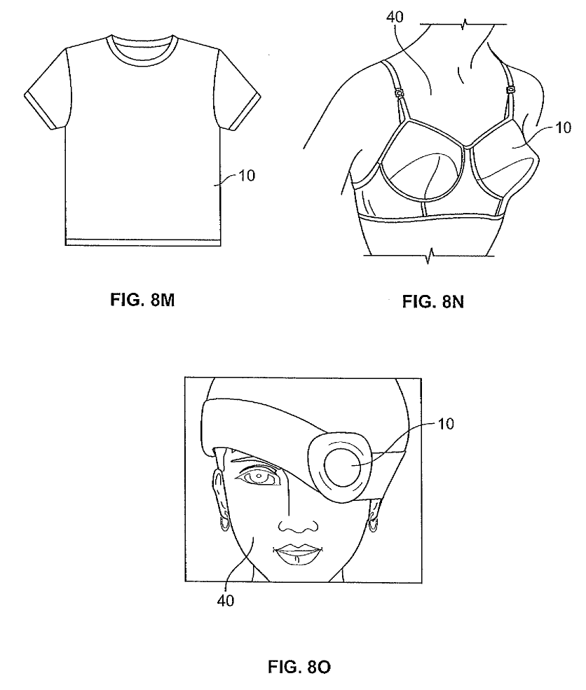

Referring now to FIGS. 8A, 8B, 8C, 8D, 8E, 8F, 8G, 8H, 8I, 8J, 8K, 8L, 8M, 8N, and 8O that depict various configurations of the light emitting medical garment device 10 showing a bandage medical garment device 10 (FIG. 8A), a blanket medical garment device 10 (FIG. 8B), a scarf medical garment device 10 (FIG. 8C), a neck brace medical garment device 10 (FIG. 8D), a back brace medical garment device 10 (FIG. 8E), a knee brace medical garment device 10 (FIG. 8F), an ankle brace medical garment device 10 (FIG. 8G), a SOC 18k medical garment device 10 (FIG. 8H), a glove medical garment device 10 (FIG. 8I), a mitten medical garment device 10 (FIG. 8J), a finger cot medical garment device 10 (FIG. 8K), a hat medical garment device 10 (FIG. 8L), a shirt medical garment device 10 (FIG. 8M), a brassiere medical garment device 10 (FIG. 8N) and an eye-patch sleeve medical garment device 10 (FIG. 8O).

As to the manner of usage and operation of the present invention, the same should be apparent from the above description. Accordingly, no further discussion relating to the manner of usage and operation will be provided.

While a preferred embodiment of the light emitting medical garment device has been described in detail, it should be apparent that modifications and variations thereto are possible, all of which fall within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification which fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, failing within the scope of the invention.

What is claimed is:

1. A light emitting medical garment device comprising:
a base;
a first bank of Light Emitting Diodes (LEDs) being attached to the base wherein the first bank of LEDs being configured to emit a first spectrum of light, the first spectrum of light being defined by a first spectral bandwidth of wavelengths and having a first predominant wavelength;
a second bank of LEDs being attached to the base wherein the second bank of LEDs being configured to emit a second spectrum of light, the second spectrum of light being defined by a second spectral bandwidth of wavelengths and having a second predominant wavelength, wherein the second predominant wavelength having an energy greater than the first predominant wavelength;
a System On a Chip (SOC) being electrically coupled to the first and second banks of LEDs; and
a power unit being operatively coupled to the SOC, and to the first and second banks of LEDs.

2. The device of claim 1 further comprising a container enshrouding the device.

3. The device of claim 1 further comprising an external housing being attached to the power unit.

4. The device of claim 1 further comprising an ON/OFF switch electrically coupled to the SOC.

5. The device of claim 1 further comprising an active agent attached to the base.

6. The device of claim 1 wherein the power unit is selected from the group consisting of a battery power unit, a high capacity capacitor power unit, a transformer power unit, and an electrical outlet plug power unit.

7. The device of claim 1 wherein the base comprises an inner pad, an outer coating, and an adhesive layer connecting the inner pad to the outer coat.

9. The device of claim 1 wherein the SOC is attached to the base.

10. The device of claim 3 wherein the SOC is attached to the external housing.

11. The device of claim 1 wherein the power unit is attached to the base.

12. The device of claim 5 wherein the active agent is selected from the group consisting of an anti-allergy active agent, an analgesic active agent, an anesthetic active agent, an antibiotic active agent, an anti-eczema active agent, an antifungal active agent, an anti-inflammatory active agent, an antiseptic active agent, an antiviral active agent, a disinfectant active agent, an immunosuppressant active agent, an steroidal active agent, a photosensitizer active agent, and admixtures thereof.

13. The device of claim 12 wherein the antibiotic active agent is selected from the group consisting of the antibiotic is selected from the group consisting of alborixin, amikacin, amphotericin B, ampicillin, aureofungin, bacitracin, bekanamycin,calcimycin, carbenicillin, cephalothin, chloramphenicol, chlortetracycline, chloromycetin. clindamycin, colistimethate, colistin, demeclocycline, dianemycin, dibekacin, doxycycline, econazole, etheromycin, erythromycin, gentamicin, grisorixin, ionomycin, kanamycin, laidlomycin, lasalocid, lenoremycin, lincomycin, lonomycin, lucensomycin, lymecycline, meclocycline, methyacycline, minocycline, monensin, monensin phenylurethane derivatives, mutalamycin, nalidixic Acid, narasin, natamycin, N-demethylrifampicin, neomycin, nigericin, nitrofurantoin, novobiocin, nystatin, oxytetracycline, penicillin, polyamido streptomycin, polymyxin, rifampicin, rolitetracycline, salinomycin, septamycin, streptomycin, streptolydigin, tetracycline, tobramycin, trimethoprim-sulfamethoxazole, and their pharmaceutically acceptable salts; the antifungal active agent is selected from the group consisting of 1-(4-chlorophenoxy)-l-(1H-imidazolyl)- 3,3-dimethyl-2-butanone, 6- cyclohexyl-l-hydroxy-4-methyl-2(1 H)-pyridone, I -hydroxy-4-methyl-6- (2,4,4trimethylpentyl)-2-(1 H)-pyridone, acyclovir, amorolfine, amphotericin B, azoles, benzyl peroxide, bifonazole, butenafine, butoconazole, chlorbutanol, chloroxylenol, ciclopirox, ciclopirox olaim me, cis-1-Acetyl-4-[4-[[2-(2,4-dichlorophenyl)-2-(1 H-imidazol-1-ylrnethyl)-1, 3-dioxolan-4-yl]methoxy]phenyl]piperazine, clotrimazole, clotrizole, diazoles, econazole, fluconazole, griseofulvin, isoconazole, itraconazole, itraconazole griseofulvin, ketoconazole, miconazole, nystatin, oxiconazole, povidone, saclicylic acid, saperconazole, sperconazole, sulconazole, 1 H-1 ,2,4-triazole-1-ethanol, terbinafine, terconazole, tioconazole, tolnaftate, triazoles, undecylenic acid, voriconazole and their pharmaceutically acceptable salts; the scabicide active agent is selected from the group consisting of benzyl benzoate, crotamiton and malathion and their pharmaceutically acceptable salts; the disinfectant active agent selected from the group consisting of 5-Chloro-2-(2,4-dichlorophenoxy)phenol, 1-hexadecylpyridinium chloride, N N″-bis(4-chlorophenyl)-3, 12-diimino- 2, 4, 11, 13 tetraaza tetradecane diimidamide, 1, I′-hexamethylenebis[5-(p-chlorophenyl)biguanide], camphor, centrimide, chloramine, chlorhexidine, citric acid, electron deficient quinones, ethyl alcohol, ethylenediamine tetraacetate, hydrogen peroxide, hypochlorite, isopropyl alcohol, lactic acid, menthol, o-phenylphenol, o-benzyl-o-cholorophenol, percarbonate, permanganate, persulfate, povidone-iodine, urea peroxides, xylenol, and their pharmaceutically acceptable salts; the antiviral active agent is selected from the group consisting of acyclovir, amantadine, 2-amino-1 ,9-dihydro-9-[(2-hydroxyethoxy)methyl1]6H-purin-6-one, dideoxyuridine, azothymidine, cis-1-acetyl-4-[4-[[2-(2,4-dichlorophenyl)-2-(1 H-imidazol-1-ylmethyl)-1,3 -dioxo-1 an-4-yl]methoxy]-phenyl]piperazine, didanosine, flucytosine (5FC), foscarnet, ganeiclovir, interferon, larniyudine, phenol, ribavirin, ritonavir, stayudine, vidarabine, zalcitabine, zidovudine, and their pharmaceutically acceptable salts; the anti-inflammatory active agent is selected from the group consisting of cortisone, dexamethasone, diclofenac, diflunisal, etodolac, fenoproben, flunisolide, hydrocortisone, ibuprofen, indomethacin, ketoprofen, ketorolac tromethamine, meclofenamate, mefenamic acid, mesalamine, methyl prednisolone, nabumetone, naproxen, piroxicam, rednisolone, prednisone, salsalate, salicylsalicylic acid, sulindac, triamcinoline, and their pharmaceutically acceptable salts; the anesthetic active agent selected from the group consisting of articaine, benzocaine, bupivacaine, bupivicaine, carbocaine, chloroprocaine, dibucaine, dimethylacetanilide, diphenhydramine, dyclonine, etidocaine, ethyl aminobenzoate, isobucaine, ketocaine, kinizocaine, lidocaine, lignocaine, marcaine, mepivacaine, meprylcaine, omegadiethylamino-2,6- and 4-aminobenzoic acid ethyl ester, parethoxycaine, piperocaine, prilocaine, primacaine, procaine, proparacaine, propoxycaine, pyrrocaine, rodocaine, ropivacaine, ropivacaine, tetracaine, trimecaine, xylocaine,and their pharmaceutically acceptable salts; the steroidal active agent is selected from the group consisting of acetonide, aclometasone, amcinonide, beciometasone, betamethasone, clobetasol, clobetasone, clocortolone, cortisone, desonide, desoxitnethasone, dexamethasone, diflorasone, diflucortolone, fluocinolone, fluocinonide, flumethasone, flurandrenolide, fluticasone, halcinonide, halobetasol, hydrocortisone, methylprednisolone, mometasone furoate, pivolate, prednicarbate, prednisolone, prednisone, triamcinolone and their pharmaceutically acceptable salts; the anti-eczema active agent is selected from the group consisting of calcipotriol, dithranolsalicylic acid, gamolenic acid, lithium succinate, tacalciol, tazarotene, and their pharmaceutically acceptable salts; the antiseptic active agent is selected from the group consisting of benzalkonium chloride, benzocaine, benzoic acid, benzoyl peroxide, cetrimide, cetylpyridinium, chlorbutanol, chlorhexidine, chlorhexideine gluconate, chlorocresol, chlorotetracycline, chloroxylenol, colistin, dibromopropamidine isothionate, framycetin, fusidic acid, hexachloropentane, hexachlorophene, hexetidine, hydroxyquinalone, iodine, lidocaine, methyl salicylate, metronidazole, muprocin, neomycin, nitrofurazone, phenol, polymoxin, povidone, resorcinol, silver sulfadiazine, tetracycline, triclosan, and their pharmaceutically acceptable salts; the anti-allergy active agent is selected from the group consisting of aluminum sulphate, antazoline, benzocaine, chlorbutanol, crotamiton, diphenhydramine, hydrocortisone, lignocaine, mepyramine, triclosan, and their pharmaceutically acceptable salts; the immunosuppressant active agent is selected from the group consisting of ascomycin, azathioprine, etamethasone diproprionate, betamethasone valerate, clobetasol proprionate, cyclosporin A, cyclosporin B, cyclosporin G, fluocinolone acetonide, halcinonide, halobetasol proprionate, hexachlorobenzene, hydrocortisone, hydrocortisone valerate, rapamycin, tacrolimus, triamcinolone acetonide, and their pharmaceutically acceptable salts; and the photosensitizer active agent is be selected from the group consisting of acridine dyes, Azure A, Azure B, Azure C, antroquinones, bacteriochlorins, basic fuschin, benzophorphyrins, Brilliant Green, carbon black, chlorins, courmarins, Crystal Violet, flavin dyes, indocyanine green, Janus Green, Malachite Green, methylene blue, methyl green, m-tetrahydroxyphyenyl chiorin, naphthalocyanines, neural red dye, new fuschin, N-hydroxypyridine-2-(1H)-thione, pararosaniline acetate, patent blue VF, phthalocyanines, pheophorbides, phenothiazines, porphyrins, psoralens, purpurins, quinolones, quinones, riboflavin-5-phosphate, Rose Bengal, tetrapyrroles, texaphyrins, tinetiopurpurins, toluidine dyes, tri-arylmethane dyes, verdins, and pharmaceutical acceptable salts.

14. The device of claim 1 wherein the first predominant wavelength is between about 1000 nm to about 570 nm; and the second predominant wavelength is between about 570 nm to about 350 nm.

15. The device of claim 1 wherein the first bank of LEDs are configured to emit light having a first luminescent power density between about μW/cm² to about 1 W/cm²; and the second bank of LEDs are configured to emit light having a second luminescent power density between of about 1 μW/cm² to about 1 W/cm².

16. The device of claim 1 wherein the first bank of LEDs is configured to emit light at a first predominant wavelength having a first bandwidth between about 1 nm to about 200 nm, and the second bank of LEDs is configured to emit light having a second bandwidth between about 1 nm to about 200 nm.

17. The device of claim 1 wherein the SOC is configured to direct the first bank of LEDs in accordance to a first timed on/off sequence, and the SOC is configured to direct the second bank of LEDs in accordance to a second timed on/off sequence.

18. The device of claim 1 wherein the light emitting medical garment device having a form selected from the group consisting of a bandage medical garment device, a blanket medical garment device, a scarf medical garment device, a neck brace medical garment device, a back brace medical garment device, a knee brace medical garment device, an ankle brace medical garment device, a sock medical garment device, a glove medical garment device, a mitten medical garment device, a finger cot medical garment device, a hat medical garment device, a shirt medical garment device, a brassiere medical garment device and an eye-patch sleeve medical garment device.

19. A kit for a light emitting medical garment device comprising:
a base;
a first bank of Light Emitting Diodes (LEDs) is attached to the base wherein the first bank of LEDs being configured to emit a first spectrum of light, the first spectrum of light being defined by a first spectral bandwidth of wavelengths and having a first predominant wavelength;
a second bank of LEDs is attached to the base wherein the second bank of LEDs being configured to emit a first spectrum of light, the second spectrum of light being defined by a second spectral bandwidth of wavelengths and having a second predominant wavelength, wherein the second predominant wavelength having an energy greater than the first predominant wavelength;
a System On a Chip (SOC) is electrically coupled to the first and second banks of LEDs; and
a power unit is operatively attachable to the SOC, and to the first and second banks of LEDs.

20. The kit of claim 19 further comprising a container enshrouding the kit.

21. The kit of claim 19 further comprising an external housing attachable to the power unit and attachable to the SOC.

22. The kit of claim 19 further comprising an active agent attachable to the base.

23. A method of using a kit for a light emitting medical garment device, the method comprising the steps of:
obtaining the kit comprising:
a base;
a first bank of Light Emitting Diodes (LEDs) is attached to the base wherein the first bank of LEDs being configured to emit a first spectrum of light, the first spectrum of light being defined by a first spectral bandwidth of wavelengths and having a first predominant wavelength;
a second bank of LEDs is attached to the base wherein the second bank of LEDs being configured to emit a second spectrum of light, the second spectrum of light being defined by a second spectral bandwidth of wavelengths and having a second predominant wavelength, wherein the second predominant wavelength having an energy greater than the first predominant wavelength;
a System On a Chip (SOC) is electrically coupled to the first and second banks of LEDs;
a container is enshrouding the base, and the first and second banks of LED, and the SOC;
a power unit is operatively attachable to the SOC, and to the first and second banks of LEDs; and
an active agent attachable to the base;
opening up the container enshrouding the base, and the first and second banks of LED, and the SOC;
removing the base and the first and second banks of LEDs and the SOC from the container;
assembling the device by coupling together the power unit to the SOC to enable the first and second banks of LEDs to emit light;
affixing a portion of the active agent either onto the base or onto a therapeutic site of a patient; and
mounting the device onto the therapeutic site of the patient.
